(19) United States
(12) Patent Application Publication
(10) Pub. No.: US 2010/0075298 A1
(43) Pub. Date: Mar. 25, 2010

(54) Method for rapid identification and quantification of microorganisms

(75) Inventors: Kathryn L. Creek, Seattle, WA (US); Alonso Castro, Santa Fe, NM (US); Perry C. Gray, Los Alamos, NM (US)

Correspondence Address: LOS ALAMOS NATIONAL SECURITY, LLC, LOS ALAMOS NATIONAL LABORATORY, PPO. BOX 1663, LC/IP, MS A187, LOS ALAMOS, NM, 87545, US

(73) Assignee: The Regents of the University of California

(21) Appl. No.: 12/284,632

(22) Filed: Sep. 23, 2008

(51) Int. Cl. C12Q 1/70; C12Q 1/68

(52) U.S. Cl. 435/5; 435/6

(57) ABSTRACT

Method for unamplified, selective identification of a microorganism comprising obtaining a sample comprising the microorganism, wherein the sample comprises unseparated genetic material of the microorganism; adding to the sample a molecular beacon comprising an nucleic sequence which is complementary to at least one nucleic acid sequence in the microorganism; and heating the sample; wherein the molecular beacon hybridizes with said nucleic acid sequence to produce a detectable signal.

### STATEMENT OF FEDERAL RIGHTS

The United States government has rights in this invention pursuant to Contract No. DE-AC52-06NA25396 between the United States Department of Energy and Los Alamos National Security, LLC for the operation of Los Alamos National Laboratory.

### FIELD OF THE INVENTION

The present invention relates to a method of identification and quantification of microorganisms, including bacteria, viruses, mold, and pollen, in liquids, on surfaces and airborne in the environment.

### BACKGROUND OF THE INVENTION

Methicillin-resistant Staphylococcus aureus (MRSA) is a serious nosocomial and community-acquired pathogen. During the last two decades, the global incidence of infections caused by this organism has increased significantly. Transmission of MRSA within and between health care institutions has been difficult to control despite strict infection control measures. Both infected and asymptomatically colonized patients may serve as reservoirs for MRSA to spread within hospitals and beyond. Large outbreaks of MRSA in a variety of institutions, such as correctional facilities, nursing homes, and among otherwise healthy individuals in the community raise the concern that this organism is spreading outside of its traditional role as a health care-related pathogen. Infections caused by MRSA result in increased lengths of hospital stay, health care costs, morbidity, and mortality compared to those caused by methicillin-sensitive strains.

Controlling the spread of MRSA requires identification of persons either colonised or infected with MRSA, followed by isolation of these persons to prevent cross-contamination, and a course of treatment. In addition, it has long been recognized that environmental surfaces in hospitals and other institutions are a major reservoir of MRSA and other multiple-resistant bacteria. To date, the most common method for identifying persons colonized or infected with MRSA relies on producing a bacterial culture of a sample, which provides results in approximately 48 to 72 hours. This length of time limits the usefulness of this method, especially with the increasingly short length of hospital stays.

Molecular identification methods are emerging as an alternative to culture. Methicillin resistance in staphylococci is encoded by the mecA gene, which is part of the staphylococcal cassette chromosome mec(SCCmec) family. Because many of the MRSA clones exhibit a heteroresistance phenotype, with only a few staphylococcal cells of the population expressing methicillin resistance, identification of the mecA gene by molecular methods has become the reference method for confirmation of MRSA strains. However, methods to detect the mecA gene lack specificity, as mecA also may be found in MR—(i.e. other methicillin-resistant bacteria), and coagulasenegative staphylococci (CoNS), such as S. epidermis, and S. haemolyticus, all of which also are commonly found in hospital settings and typically are not associated with adverse health effects. Another disadvantage of molecular identification methods is that current commercially available methods fail to provide quantitative information; rather, they provide at best only semi-quantitative information, and in most instances only indicate whether the pathogen is present.

Finally, the many molecular methods for MRSA are based on polymerase chain reaction (PCR) technology, which uses primers that target the relevant genes. PCR-based identification is time-consuming, labor intensive, prone to sample contamination, and currently are not amenable to use in the field or high-throughput automation. In addition, multiplexed PCR methods that target both mecA and nuc genes cannot be applied to the direct identification of MRSA from nonsterile specimens such as nasal swabs or from environmental surfaces, as these samples often contain a mixture of CoNS and S. aureus. Since either of these organisms can carry mecA, both the mecA and nuc genes need to be simultaneously detected within the same bacterium to avoid a false-positive result.

A need exists, therefore, for a method for the identification of MRSA and other pathogenic organisms which is suitable for use in commercial settings such as hospitals and other institutions, i.e., a method which is rapid, selective, quantitative, unamplified (non-PCR based), cost-effective and amenable to high-throughput automation.

### SUMMARY OF THE INVENTION

The present invention meets the aforementioned needs by providing a method for identification of microorganisms which utilizes molecular beacons in combination with ultra-sensitive fluorescence detection. The method is believed to be useful for selective and simultaneous identification of a multitude of other microorganisms, both bacterial and viral.

The molecular beacons have nucleic acid sequences specifically designed to be complementary to the microorganism of interest (one example of which is MRSA mecA and nuc mRNAs), and thus capable of hybridizing to a variety of microorganisms. Previously, it was not thought that identification of microorganisms could be performed without the time-consuming process of lysing cells, extracting DNA, and quantitating the DNA itself (which can then be roughly correlated to the number of microorganisms present). Thus, the idea that identification of microorganisms could be performed rapidly and in the field—possibly even with a hand-held unit—was rejected by those of skill in the art, as these steps require a well-equipped laboratory setting. The present invention does not require DNA extraction and amplification, and allows identification and quantification of intact microorganisms. Thus, the present invention allows direct quantification of the number of microorganisms, including identification of a single microorganism by imaging and counting individual cells. In addition, use of molecular beacons with intact cells and spores means that the method is less time-consuming and suitable for continuous, real-time identification.

Another advantage of the present invention is that it allows distinction between living and dead organisms. Thus, for example, if an area is believed to be infected and is treated with an anti-microbial agent, it is important to be able to tell if the agent was effective and what amount, if any, of the living microorganism remains.

Molecular beacons may comprise in one embodiment, nucleic acid molecules having a target complementary sequence, an affinity pair (or nucleic acid arms) holding the probe in a closed conformation in the absence of a target nucleic acid sequence, and a label pair that interacts when the probe is in a closed conformation. In another embodiment, hybridization of the target nucleic acid and the target complementary sequence separates the members of the affinity pair, thereby shifting the probe to an open conformation. The shift to the open conformation is induced by heating, and is detectable due to reduced interaction of the label pair, for example, a fluorophore and a quencher (e.g., DABCYL™ and EDANS™). Non-limiting examples of molecular beacons are fully described in U.S. Pat. No. 5,925,517.

The molecular beacons comprise, in one embodiment, a fluorescent label which fluoresces upon hybridization and can be detected using an ultrasensitive fluorescence imager. This allows the bacteria of interest to be individually detected and quantitated (“single-microorganism detection”), and therefore the method is highly sensitive and quantitative. Because the molecular beacons bind to specific genes within a microorganism, the method is highly selective. In addition, the method is suitable for incorporation into an apparatus to simplify its use. The method does not require extraction of genetic material (DNA or RNA) and is not based on PCR technology; therefore the method of the present invention is rapid and provides real-time qualitative analysis, is suitable for high throughput automation, and is significantly less expensive than currently available methods. Finally, in contrast to other methods, the method of the present invention may be used to analyze samples obtained from air, on surfaces, and in human biological fluids, and is non-destructive, which allows for confirmation of results by other laboratory methods.

The following describe some non-limiting embodiments of the present invention.

According to one embodiment of the present invention, a method is provided for unamplified, selective identification of a microorganism comprising obtaining a sample comprising the microorganism, wherein the sample further comprises unseparated genetic material of the microorganism; adding to the sample a molecular beacon comprising a nucleic sequence which is complementary to at least one nucleic acid sequence in the microorganism; and heating the sample; wherein the molecular beacon hybridizes with said nucleic acid sequence to produce a detectable signal.

According to another embodiment of the present invention, a method for unamplified, selective identification of methicillin-resistant Staphylococcus aureus is provided comprising: obtaining a sample comprising methicillin-resistant Staphylococcus aureus, wherein the sample comprises unseparated genetic material of the methicillin-resistant Staphylococcus aureus; adding to the sample a molecular beacon comprising a nucleic acid sequence selected from the group consisting of GCG GCT GCT GGC ACG TAG TTA (SEQ ID NO: 1), CGC GAT TIC AAT ATG TAT GCT TTG GTC TTT CTG ATC GCG (SEQ ID NO: 3), and combinations thereof; and heating the sample; wherein the molecular beacon hybridizes a nucleic acid sequence in the organism of interest to produce a detectable signal.

### BRIEF DESCRIPTION OF THE DRAWINGS

FIG. 1 depicts the fluorescence intensity (y-axis) measured as a function of wavelength, of a molecular beacon and a suspension of untreated B. subtilis spores compared to the signal obtained from the molecular beacon alone.

### DETAILED DESCRIPTION OF THE INVENTION

In all embodiments of the present invention, all percentages are by weight of the total composition, unless specifically stated otherwise. All ratios are weight ratios, unless specifically stated otherwise. All ranges are inclusive and combinable. The number of significant digits conveys neither a limitation on the indicated amounts nor on the accuracy of the measurements. All numerical amounts are understood to be modified by the word “about” unless otherwise specifically indicated. All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

As used herein, “unamplified” means that the method does not require replication of nucleic acid sequences for identification. One non-limiting example of replication is PCR-based technologies.

As used herein, “unseparated” means that genetic material such as DNA and RNA is not removed or substantially isolated from a microorganism and/or cellular components for further amplification or analysis. “Unseparated” is not intended to imply that the sample is not lysed; the sample may or may not be lysed.

As used herein, “identification” means identifying the type of microorganism and/or strain of microorganism, and is distinguished from “detection,” which is understood to mean that determination of the presence of a microorganism without necessarily determination of the specific type of microorganism and/or strain.

As used herein, “selective identification” means the identification of an organism of interest in a sample comprising at least one additional organism having one or more nucleic acid sequences in common with the organism of interest, wherein the additional organism(s) do not produce a signal which is stronger than the normally present background signal. One example of selective identification is identification of MRSA in a sample comprising MR—(i.e. other methicillin-resistant bacteria), S. epidermis, and/or S. haemolyticus

As used herein, “sample,” means a discrete quantity of a medium intended for subsequent analysis, which may or may not contain a microorganism of interest. The sample may be obtained, for example, from the air (ambient environment), a filter, a surface, a wipe or swab, a bodily fluid, and/or directly from a human, animal or plant.

As used herein, “molecular beacon” means a molecule comprising nucleic acid molecules having a target complementary sequence, an affinity pair (or nucleic acid arms) holding the molecule in a closed conformation (e.g., the nucleic acid molecules forming a loop) in the absence of a target nucleic acid sequence, and a label pair that interacts when the probe is in a closed conformation and emits a signal when the probe is in an open conformation.

As used herein, “detectable signal” means a signal originating from the molecular beacon which is detectable using appropriate instrumentation as would be understood by one of skill in the art, and which produces a signal that is greater than a signal produced by a suitable control sample not comprising the organism of interest, on the same instrument. Alternatively, “detectable signal” may mean a signal that is visible to a human having unimpaired vision.

As used herein, “ultrasensitive fluorescence imager” means an instrument capable of detection of fluorescence emission from a single microorganism.

As used herein, “correlates” means that a signal is mathematically related to a concentration of the microorganism of interest, typically by means of a calibration curve curve or use of a mathematical equation using particle count and sample volume, as would be readily understood by one of skill in the art.

As used herein, “surface” means any substrate whereon a microorganism of interest may be found, non-limiting examples of which include a counter top, table top, floor, bedding, clothing, or an apparatus.

The present method describes a method for unamplified, selective identification of a microorganism of interest. By “microorganism” is meant an organism which comprises at least one nucleic acid sequence. In one embodiment, the microorganism is substantially intact, meaning that the microorganism has not been subjected to cell lysis. Alternatively, the microorganism may be subjected to lysis. The method may be useful for identification of a wide variety of microorganisms, including bacteria, viruses, mold, fungi, protozoan parasites, and combinations thereof. Non-limiting examples of bacterial microorganisms include Staphylococcus aureus, methicillin-resistant Staphylococcus aureus (MRSA), vanomycin-resistant Staphylococcus aureus, vanomycin-intermediate Staphylococcus aureus, Clostridium difficile, Acinobacter baumanni, vanomycin-resistant enterococci (VRE), gram negative bacilli (GNB), Streptococcus pneaumoniae, Salmonella enteritidis, Salmonella typhimurium, Serratia Bacillus anthracis, Yersinia pestis, Francisella tularensis, Xylella fastidiosa, Aphtae epizooticae, Burkholderia mallei, and combinations thereof. Non-limiting examples of viral microorganisms include influenza virus, coronavirus, malaria, ebola virus, foot and mouth virus, SARS coronavirus, and combinations thereof. In one embodiment, the microorganism of interest is methicillin-resistant Staphylococcus aureus.

The method comprises adding to a sample a molecular beacon comprising a nucleic sequence which is complementary to at least one nucleic acid sequence in the microorganism of interest. Molecular beacons are described generally in U.S. Pat. No. 5,925,517, and in “Wavelength-shifting molecular beacons”. S. Tyagi, Nature Biotechnology 18(11), 1191-96 (2000). Upon contact with the microorganism of interest, the nucleic acid sequence of the molecular beacon hybridizes (i.e., forms covalent bonds with the nucleic acids of the microorganism, thereby fusing with the DNA or RNA of the microorganism). Upon hybridization, the molecular beacon produces a first signal, which originates from a first signaling molecule attached to the molecular beacon. The first signal is suitable for detection with a variety of analytical instrumentation. Hybridization may occur with nucleic acids on the cellular surface, intracellular nucleic acids, or both.

In one embodiment, two or more molecular beacons may be used, each containing a different nucleic acid sequence. The nucleic acids may be capable of hybridizing with more than one nucleic acid sequence of the same type of microorganism, or may be capable of hybridizing with a nucleic acid sequence of multiple types of microorganisms. In one embodiment, at least two, and alternatively at least five, and alternatively at least ten, and alternatively from 2 to 10 molecular beacons may be used, where each molecular beacon comprises a different nucleic acid sequence are used.

The molecular beacons of the present invention have been synthesized to incorporate nucleic acid sequences uniquely suited for identification of the microorganism of interest. In one embodiment, the nucleic acid sequence is suitable for the identification of methicillin-resistant Staphylococcus aureus and is GCG GCT GCT GGC ACG TAG TTA (SEQ ID NO: 1). Other non-limiting examples of nucleic acid sequences suitable for use in the molecular beacons of the present invention include one or more of the following:

CCC GCG CGT AGT TAC TGC GTT GTA AGA CGT CCG CGG G (SEQ ID NO: 2), suitable for identification and/or quantitation of Staphylococcus aureus;

CGC GAT TTC AAT ATG TAT GCT TTG GTC TTT CTG ATC GCG (SEQ ID NO: 3), suitable for identification and/or quantitation of MRSA;

CAC GCG GAT TTT GAA TCT CTT CCT CTA GTA GCG CGT G (SEQ ID NO: 4), suitable for identification and/or quantitation of Clostridium difficile;

GCG GAT AGT GTG ATC TGA CGA AGA CAC ATT AAC TAT CGC G (SEQ ID NO: 5), suitable for identification and/or quantitation of Acinetobacter baumannii;

CGC GAT TCG ATG AGG GCG GAA AAC CCA ATA ATT ATC GCG (SEQ ID NO: 6), suitable for identification and/or quantitation of Vancomycin-resistant enterococci (VRE);

CGC GAT CAG GTC TCA GCA TTC CAA CCG CCG ATC GCG (SEQ ID NO: 7), suitable for identification and/or quantitation of Streptococcus pneumoniae;

CGC TAT CCG GGG CGT AAC CCG TAG CG (SEQ ID NO: 8), suitable for identification and/or quantitation of Salmonella typhimurium;

CGC GAT TGC GCT TTA CGC CCA GTA ATT CCG AAT CGC G (SEQ ID NO: 9), suitable for identification and/or quantitation of Serratia marcescens;

CCG TCA CTG GGA GAA AGA AAT GGT AGG TTG TTG GAA TGA CGG (SEQ ID NO: 10), suitable for identification and/or quantitation of Chlamydia trachomatis;

CCT GGA GCC GAC TGT TGG CGC TGT CCA GG (SEQ ID NO: 11), suitable for identification and/or quantitation of multi-drug resistant Mycobacterium tuberculosis;

CCA AGC TAA GAY CGT TTG GTG CCT TGG CTT GG (SEQ ID NO: 12), where Y is an unspecified pyrimidine base, suitable for identification and/or quantitation of influenza A virus;

CGC GAT AAC AGC CTG AAG GAA GCA ACG AAA TCG CG (SEQ ID NO: 13), suitable for identification and/or quantitation SARS coronavirus;

CTG GCC CTG GAG GAG TAT TAA TGT TAT TAT CGG CCA G (SEQ ID NO: 14), suitable for identification and/or quantitation of Plasmodium falciparum, a malarial protozoan parasite;

CGC TAT GGT GAA GGT GGA ATG GTT GTC ACG AAT AGC G (SEQ ID NO: 15), suitable for identification and/or quantitation of E. coli O157:H7;

CGC GAT CAG ACG AAG TGG TCA TGC TTG ATC GCG (SEQ ID NO: 16), suitable for identification and/or quantitation of Enterobacter aerogenes;

CCG ACG AGG GTT GTC AGA GGA TGC GTC GG (SEQ ID NO: 17), suitable for identification and/or quantitation of Bacillus anthracis;

CGC GAT TGT CTG TTT CCC ATA GAT GCC ATG AAT CGC G (SEQ ID NO: 18), suitable for identification and/or quantitation of Yersinia pestis;

CGC TCGTGG AGT CGG TGT AAA GGC TCC GAG CG (SEQ ID NO: 19), suitable for identification and/or quantitation of Francisella Tularensis;

CGC GAT CTA CCA GCA GCG CCA GAC GGA TCG CG (SEQ ID NO: 20), suitable for identification and/or quantitation of Ebolavirus;

CGC GAT ATC CTC TCC TTT GCA CGC CGT GGG ACC ATA TCG CG (SEQ ID NO: 21), suitable for identification and/or quantitation of Aphtae epizooticae (virus causing foot and mouth disease);

CGC GAT AAC CGC AGC AGA AGC CGC TCA TCA TCG CG (SEQ ID NO: 22), suitable for identification and/or quantitation of Xylella fastidiosa (bacteria causing Pierce's disease in plants);

CGC TGC GTT GGG GAT TCA TTT CCT TAG TAA GCA GCG (SEQ ID NO: 23), suitable for identification of Burkholderia mallei (Glander's disease); and

CGC GAT CTA ACT ACG TGC CAG CAG CCG CGA TCG CG (SEQ ID NO: 24), suitable for identification of Bacillus subtilus.

In one embodiment, the molecular beacons comprise a nucleic acid sequence selected from the group consisting of GCG GCT GCT GGC ACG TAG TTA (SEQ ID NO: 1); CGC GAT TTC AAT ATG TAT GCT TTG GTC TTT CTG ATC GCG (SEQ ID NO: 3); CGC GAT CAG GTC TCA GCA TTC CAA CCG CCG ATC GCG (SEQ ID NO: 7); CCG TCA CTG GGA GAA AGA AAT GGT AGG TTG TTG GAA TGA CGG (SEQ ID NO: 10); CCA AGC TAA GAY CGT TTG GTG CCT TGG CTT GG (SEQ ID NO: 12), where Y is an unspecified pyrimidine base; CGC GAT AAC AGC CTG AAG GAA GCA ACG AAA TCG CG (SEQ ID NO: 13); CGC TAT GGT GAA GGT GGA ATG GTT GTC ACG AAT AGC G (SEQ ID NO: 15); CGC GAT CAG ACG AAG TGG TCA TGC TTG ATC GCG (SEQ ID NO: 17); CGC GAT CTA CCA GCA GCG CCA GAC GGA TCG CG (SEQ ID NO: 20); CGC GAT ATC CTC TCC TTT GCA CGC CGT GGG ACC ATA TCG CG (SEQ ID NO: 21); CGC GAT CTA ACT ACG TGC CAG CAG CCG CGA TCG CG (SEQ ID NO: 24), and combinations thereof.

In yet another embodiment, the molecular beacons comprise a nucleic acid sequence selected from the group consisting of GCG GCT GCT GGC ACG TAG TTA (SEQ ID NO: 1); CGC GAT TTC AAT ATG TAT GCT TTG GTC TTT CTG ATC GCG (SEQ ID NO: 3); CCA AGC TAA GAY CGT TTG GTG CCT TGG CTT GG (SEQ ID NO: 12), where Y is an unspecified pyrimidine base; CGC GAT AAC AGC CTG AAG GAA GCA ACG AAA TCG CG (SEQ ID NO: 13); CGC GAT CAG ACG AAG TGG TCA TGC TTG ATC GCG (SEQ ID NO: 17); CGC GAT CTA CCA GCA GCG CCA GAC GGA TCG CG (SEQ ID NO: 20); CGC GAT CTA ACT ACG TGC CAG CAG CCG CGA TCG CG (SEQ ID NO: 24); and combinations thereof.

In yet another embodiment, the molecular beacons comprise a nucleic acid sequence suitable for identification and/or quantitation of a bacteria, and are selected from the group consisting of GCG GCT GCT GGC ACG TAG TTA (SEQ ID NO: 1); CCC GCG CGT AGT TAC TGC GTT GTA AGA CGT CCG CGG G (SEQ ID NO: 2); CGC GAT TTC AAT ATG TAT GCT TTG GTC TTT CTG ATC GCG (SEQ ID NO: 3); CAC GCG GAT TTT GAA TCT CTT CCT CTA GTA GCG CGT G (SEQ ID NO: 4); GCG GAT AGT GTG ATC TGA CGA AGA CAC ATT AAC TAT CGC G (SEQ ID NO: 5); CGC GAT TCG ATG AGG GCG GAA AAC CCA ATA ATT ATC GCG (SEQ ID NO: 6); CGC GAT CAG GTC TCA GCA TTC CAA CCG CCG ATC GCG (SEQ ID NO: 7); CGC TAT CCG GGG CGT AAC CCG TAG CG (SEQ ID NO: 8); CGC GAT TGC GCT TTA CGC CCA GTA ATT CCG AAT CGC G (SEQ ID NO: 9); CCG TCA CTG GGA GAA AGA AAT GGT AGG TTG TTG GAA TGA CGG (SEQ. ID NO: 10); CCT GGA GCC GAC TGT TGG CGC TGT CCA GG (SEQ ID NO: 11); CGC TAT GGT GAA GGT GGA ATG GTT GTC ACG AAT AGC G (SEQ ID NO: 15); CGC GAT CAG ACG AAG TGG TCA TGC TTG ATC GCG (SEQ ID NO: 16); CGC GAT CAG ACG AAG TGG TCA TGC TTG ATC GCG (SEQ ID NO: 17): CGC GAT TGT CTG TTT CCC ATA GAT GCC ATG AAT CGC G (SEQ ID NO: 18); CGC TCG TGG AGT CGG TGT AAA GGC TCC GAG CG (SEQ ID NO: 19); CGC GAT AAC CGC AGC AGA AGC CGC TCA TCA TCG CG (SEQ ID NO: 22); CGC TGC GTT GGG GAT TCA TTT CCT TAG TAA GCA GCG (SEQ ID NO: 23); CGC GAT CTA ACT ACG TGC CAG CAG CCG CGA TCG CG (SEQ ID NO: 24); and combinations thereof.

In yet another embodiment, the molecular beacons comprise a nucleic acid sequence suitable for identification and/or quantitation of a virus, and are selected from the group consisting of CCA AGC TAA GAY CGT TTG GTG CCT TGG CTT GG (SEQ ID NO: 12); CGC GAT AAC AGC CTG AAG GAA GCA ACG AAA TCG CG (SEQ ID NO: 13); CGC GAT CTA CCA GCA GCG CCA GAC GGA TCG CG (SEQ ID NO: 20); CGC GAT ATC CTC TCC TTT GCA CGC CGT GGG ACC ATA TCG CG (SEQ ID NO: 21); and combinations thereof.

The sample may be obtained from a variety of sources, including but not limited to the air, a liquid, a surface, soil, a human, an animal, a plant, or combinations thereof. Non-limiting examples of animals include wild and domestic mammals and avians. Domestic animals may include dogs, cats, sheep, horses, pigs, etc; and avians such as chickens, ducks, geese, etc. When obtained from the air, the sample may be collected onto a filter, an adhesive-coated disk, sample wheel, or other suitable collection means and subsequently brought into contact with the molecular beacon. When the sample is collected from a surface, such as a counter top, table top, fabric, or any other surface thought to be exposed to the microorganism of interest, the sample may be collected by a number of means that would be known to one of skill in the art, non-limiting examples of which include a wipe, a swab or the like. Alternatively, the sample on the surface need not be collected; rather, analysis may proceed directly on the surface. When the sample is collected from a human, the sample may be in the form of sputum, blood, plasma, urine, feces, or other suitable fluid, and may be collected by means of a swab, wipe, syringe, or other suitable means that would be known to one of skill in the art.

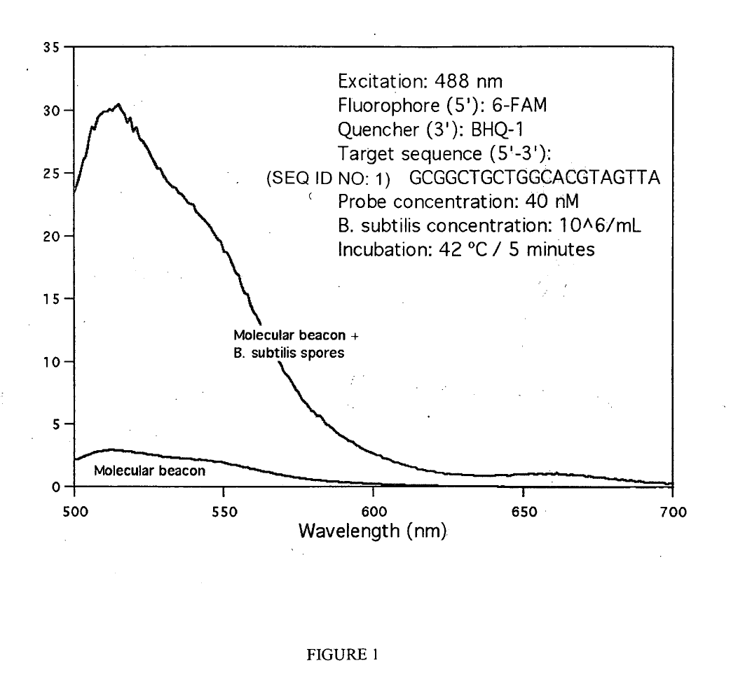

The method of the present invention comprises the step of heating the sample, which allows the molecular beacon to preferentially bind to the target as depicted in FIG. 1. The temperature to which the sample may be heated will vary, depending upon the type of molecular beacon, and determination of the appropriate temperature would be apparent to one of skill in the art. In one embodiment, the sample is heated to a temperature of from about 35° C. to about 45° C.

The method of the present invention may comprise determining whether the microorganism of interest is viable (i.e., is alive or dead). Viability is an indicator of risk of disease or illness, and as a pre- and a post-testing procedure to confirm decontamination. The viability may be assessed by using a second signaling molecule, which produces a second detectable signal suitable for identification with a variety of analytical instrumentation. Thus, the presence of a second detectable signal is an indication of viability, whereas the first detectable signal is suitable for detection, identification and quantitation. One non-limiting example of a means for determining viability is BacLight™ Bacterial Viability Kits, and LIVE/DEAD® Fixable Dead Cell Stain Kit, both commercially available from Molecular Probes® (Invitrogen®, Carlsbad Calif.).

The signal produced by the molecular beacon upon hybridization may comprise a fluorescence signal. The signal may be used to quantitate the amount of the microorganism of interest which is present in the sample. In one embodiment, the fluorescence signal is detected by a fluorescence imager, one non-limiting example of which is described in “Detection, enumeration, and sizing of planktonic bacteria by image-analyzed epifluorescence microscopy” M E Sieracki, P W Johnson, and J M Sieburth, Appl Environ Microbiol. 49, 799-810 (1985). In one embodiment, the fluorescence imager is capable of single molecule detection. Preferably, the instrument is small, lightweight and field-portable. Quantitation may occur by a variety of means that would be understood by one of skill in the art, for example, by constructing a calibration curve which measures an output signal from the instrument produced by two or more compositions having a known concentration of analyte, fitting an equation such as that representing a line to the data obtained from the signals, and utilizing the resulting known relationship to analyze an unknown concentration.

In one embodiment, quantitation may comprise providing a filter, said filter comprising a molecular beacon suitable for use with a specific microorganism; exposing the filter to the microorganism; removing the microorganism and attached molecular beacon from the disk, and quantitating the number of microorganisms. One non-limiting example of a suitable filter is an adhesive-coated disk, which may form part of a disc- or wheel-type rotatable sampler. One method of removing the microorganism and attached molecular beacon from the filter is to use an adhesive to which pressure is applied to adhere the microorganism and attached molecular beacon, and subsequently removing the adhesive. One non-limiting method of quantitating the number of microorganism is to perform fluorescence imaging and count the number of molecular beacons that emit a fluorescence signal.

### EXAMPLE

### Example 1

A molecular beacon with sequence 5′-CGCGATCTAACTACGTGCCAGCAGCCGCGATCGCG-3′ (SEQ ID NO: 24) that targets the rmH-16S gene of Bacillus subtilus was obtained from Sigma-Aldrich. The self-complementary stem sequence is shown undelined above. This molecular beacon is labeled with 6-carboxyfluorescein (6-FAM) at the 5′ end and with the quencher BHQ-1™ at the 3′ end. The molecular beacon was mixed with untreated Bacillus subtilis spores at a final concentration of 40 nM and 10⁶ spores/mL respectively, in a final volume of 5 mL of standard Tris-EDTA buffer. After mixing, the mixture was incubated at 42° C. for about 5 minutes to ensure binding of the molecular beacon molecules to the specific-sequence target DNA molecules present on the surface of the spores.

The fluorescence spectrum of the hybridization mixture was obtained with a Shimadzu 1501 spectrofluorophotometer in a 1 cm quartz cell, using 488 nm excitation light. The spectrum is shown in FIG. 1, which also shows the fluorescence spectrum of a 40 nM molecular beacon solution. Without wishing to be limited by theory, it is believed that the large difference in fluorescence intensity is due to the binding of the molecular beacon to the specific-sequence DNA target of Bacillus subtilis, which causes the 6-FAM molecule to be unquenched and to fluoresce.

The hybridization mixture was observed with a Zeiss epifluorescent microscope at 400× magnification using blue light excitation. The presence of individual, discrete, fluorescent spores having an average diameter of approximately 1 μm indicates that the DNA being targeted is contained as part of the individual Bacillus subtilis spores, and is not free in solution.

### Example 2

Molecular beacons having the following sequences in Table 1 may be used in the procedure outlined in Example 1 to target the corresponding microorganism of interest. Note that the temperature to which the mixture is heated may vary according to manufacturer's instructions and to the knowledge available to one of skill in the art.

| | | |
|---|---|---|
| Microorganism | Target Gene | Sequence (5′-3′) |

| | | |
|---|---|---|
| Methycillin-resistant Staphylococcus | | GCG GCT GCT GGC ACG TAG TTA |
| aureus | | (SEQ ID NO: 1) |
| Staphylococcus aureus | orfX | CCC GCG CGT AGT TAC TGC GTT GTA AGA CGT CCG CGG G |
| | | (SEQ ID NO: 2) |
| Methycillin-resistant Staphylococcus | mecA | CGC GAT TTC AAT ATG TAT GCT TTG GTC TTT CTG ATC GCG |
| aureus | | (SEQ ID NO: 3) |
| Clostridium difficile | tcdA | CAC GCG GAT TTT GAA TCT CTT CCT CTA GTA GCG CGT G |
| | | (SEQ ID NO: 4) |
| Acinetobacter baumannii | 16S-23S intergenic | GCG GAT AGT GTG ATC TGA CGA AGA CAC ATT AAC TAT CGC G |
| | region | (SEQ ID NO: 5) |
| Vanomycin-resistant enterococci | vanA | CGC GAT TCG ATG AGG GCG GAA AAC CCA ATA ATT ATC GCG |
| (VRE) | | (SEQ ID NO: 6) |
| Streptococcus pneumoniae | lytA | CGC GAT CAG GTC TCA GCA TTC CAA CCG CCG ATC GCG |
| | | (SEQ ID NO: 7) |
| Salmonella typhimurium | himA | CGC TAT CCG GGG CGT AAC CCG TAG CG |
| | | (SEQ ID NO: 8) |
| Serratia marcescens | 16s rRNA | CGC GAT TGC GCT TTA CGC CCA GTA ATT CCG AAT CGC G |
| | | (SEQ ID NO: 9) |
| Chlamydia trachomatis | KL1/KL2 | CCG TCA CTG GGA GAA AGA AAT GGT AGG TTG TTG GAA TGA |
| | | CGG |
| | | (SEQ ID NO: 10) |
| Multi-drug resistant Mycobacterium | rpoB | CCT GGA GCC GAC TGT TGG CGC TGT CCA GG |
| tuberculosis | | (SEQ ID NO: 11) |
| Influenza A virus | nucleoprotein | CCA AGC TAA GAY CGT TTG GTG CCT TGG CTT GG |
| | | (SEQ ID NO: 12) |
| SARS coronavirus | N/A | CGC GAT AAC AGC CTG AAG GAA GCA ACG AAA TCG CG |
| | | (SEQ ID NO: 13) |
| Plasmodium falciparum | cox1 | CTG GCC CTG GAG GAG TAT TAA TGT TAT TAT CGG CCA G |
| | | (SEQ ID NO: 14) |
| Escherichia coli O157: H7 | rfbE | CGC TAT GGT GAA GGT GGA ATG GTT GTC ACG AAT AGC G |
| | | (SEQ ID NO: 15) |
| Enterobacter aerogenes | marA | CGC GAT CAG ACG AAG TGG TCA TGC TTG ATC GCG |
| | | (SEQ ID NO: 16) |
| Bacillus anthracis | 16S rRNA | CGC GAT CAG ACG AAG TGG TCA TGC TTG ATC GCG |
| | | (SEQ ID NO: 17) |
| Yersinia pestis | PIM | CGC GAT TGT CTG TTT CCC ATA GAT GCC ATG AAT CGC G |
| | | (SEQ ID NO: 18) |
| Francisella Tularensis | 16S rRNA | CGC TCG TGG AGT CGG TGT AAA GGC TCC GAG CG |
| | | (SEQ ID NO: 19) |
| Ebolavirus | GP | CGC GAT CTA CCA GCA GCG CCA GAC GGA TCG CG |
| | | (SEQ ID NO: 20) |
| Aphtae epizooticae | N/A | GCG GAT ATC CTC TCC TTT GCA CGC CGT GGG ACC ATA TCG CG |
| | | (SEQ ID NO: 21) |
| Xylella fastidiosa | N/A | CGC GAT AAC CGC AGC AGA AGC CGC TCA TCA TCG CG |
| | | (SEQ ID NO: 22) |
| Burkholderia mallei | 16S rRNA | CGC TGC GTT GGG GAT TCA TTT CCT TAG TAA GCA GCG |
| | | (SEQ ID NO: 23) |

### Example 3

The following describes one example of a suitable sample collector, i.e. a bioaerosol detector.

A bioaerosol detector may be activated either manually or by means of a bioaerosol-detecting trigger. The sample collection plate comprises 96 individually addressable wells containing a thin layer of liquid silicone which aids in the collection of impacted particles. Particles greater then the filter cutoff size are deposited on the substrate. A standard filtration device or system could be used consisting of a filter or multiple filters. After collecting approximately 10-20 liters of air in one of the wells by means of a high efficiency DC diaphragm pump (70 liter.min. maximum flow), the well is treated with an appropriate composition comprising a suitable molecular beacon, as described in Examples 1 and 2, and fluorescence at the appropriate band is measured immediately by a two-axis positionable, miniaturized epifluorescence detector, such as those commonly found in microtiter plate readers. The bioaerosol detector can be configured to operate in one of three different modes of operation, which is determined by the composition of the taggant mixture:

(i) Bioaerosol detection mode. The taggant mixture contains a suitable intercalator dye, which will fluoresce if nucleic-acid-containing particles have been collected in the well. Since only small amounts of sample are required for obtaining a positive bioaerosol signal, this mode of operation is suitable for low-risk environments when continuous monitoring of bioaerosol levels is required.
(ii) Agent detection mode. The taggant mixture contains a cocktail of 13 molecular beacon probes, which cover 31 microorganisms. If one or more of these pathogens is present, the instrument produces a positive detect signal, which is conducive to evacuation measures. This mode of operation is suitable for discriminating between threat agents and ordinary background microorganisms.
(iii) Agent identification. The taggant mixture in a specific well contains a molecular beacon that is specific for a unique threat agent.
(iv) Hybrid mode. The 96-well plate is divided into sections containing molecular beacons that correspond to a combination of the three previous modes of operation. The actual sampling sequence is determined by the particular threat scenario, which can be pre-programmed via software.

It is important to note that the above mixture does not require the handling of liquid, since each of the 96 wells in a plate is preloaded with the appropriate molecular beacon. This configuration avoids the need to remove a filter, use a liquid for extraction, or place drops of an extracted sample in liquid on a ticket.

Whereas particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method for unamplified, selective identification of a microorganism comprising:
a) obtaining a sample comprising the microorganism, wherein the sample comprises unseparated genetic material of the microorganism;
b) adding to the sample a molecular beacon comprising an nucleic sequence which is complementary to at least one nucleic acid sequence in the microorganism; and
c) heating the sample;
wherein the molecular beacon hybridizes with said nucleic acid sequence to produce a detectable signal.

2. The method of claim 1, wherein the sample is obtained from the air, a liquid, a surface, soil, a human, an animal, a plant, or combinations thereof.

3. The method of claim 1, wherein the microorganism is selected from the group consisting of bacteria, viruses, mold, fungi, protozoan parasites, and combinations thereof.

4. The method of claim 1, wherein the microorganism is a bacteria.

5. The method of claim 4, wherein the nucleic acid sequence is selected from the group consisting of GCG GCT GCT GGC ACG TAG TTA (SEQ ID NO: 1); CCC GCG CGT AGT TAC TGC GTT GTA AGA CGT CCG CGG G (SEQ ID NO: 2); CGC GAT TTC AAT ATG TAT GCT TTG GTC TTT CTG ATC GCG (SEQ ID NO: 3); CAC GCG GAT TTT GAA TCT CTT CCT CTA GTA GCG CGT G (SEQ ID NO: 4); GCG GAT AGT GTG ATC TGA CGA AGA CAC ATT AAC TAT CGC G (SEQ ID NO: 5); CGC GAT TCG ATG AGG GCG GAA AAC CCA ATA ATT ATC GCG (SEQ ID NO: 6); CGC GAT CAG GTC TCA GCA TTC CAA CCG CCG ATC GCG (SEQ ID NO: 7); CGC TAT CCG GGG CGT AAC CCG TAG CG (SEQ ID NO: 8); CGC GAT TGC GCT TTA CGC CCA GTA ATT CCG AAT CGC G (SEQ ID NO: 9); CCG TCA CTG GGA GAA AGA AAT GGT AGG TTG TTG GAA TGA CGG (SEQ ID NO: 10); CCT GGA GCC GAC TGT TGG CGC TGT CCA GG (SEQ ID NO: 11); CGC TAT GGT GAA GGT GGA ATG GTT GTC ACG AAT AGC G (SEQ ID NO: 15); CGC GAT CAG ACG AAG TGG TCA TGC TTG ATC GCG (SEQ ID NO: 16); CGC GAT CAG ACG AAG TGG TCA TGC TTG ATC GCG (SEQ ID NO: 17): CGC GAT TGT CTG TTT CCC ATA GAT GCC ATG AAT CGC G (SEQ ID NO: 18); CGC TCG TGG AGT CGG TGT AAA GGC TCC GAG CG (SEQ ID NO: 19); CGC GAT AAC CGC AGC AGA AGC CGC TCA TCA TCG CG (SEQ ID NO: 22); CGC TGC GTT GGG GAT TCA TTT CCT TAG TAA GCA GCG (SEQ ID NO: 23); CGC GAT CTA ACT ACG TGC CAG CAG CCG CGA TCG CG (SEQ ID NO: 24);
and combinations thereof.

6. The method of claim 4, wherein the bacteria is methicillin-resistant Staphylococcus aureus and the nucleic acid sequence comprises GCG GCT GCT GGC ACG TAG TTA (SEQ ID NO: 1), CGC GAT TTC AAT ATG TAT GCT TTG GTC TTT CTG ATC GCG (SEQ ID NO: 3), or combinations thereof.

7. The method of claim 4, wherein the bacteria is Bacillus subtilus and the nucleic acid sequence comprises CGCGATCTAACTACGTGCCAGCAGCCGCGATCGCG-3′ (SEQ ID NO: 24).

8. The method of claim 4, wherein the bacteria is Bacillus anthracis and the nucleic acid sequence comprises CGC GAT CAG ACG AAG TGG TCA TGC TTG ATC GCG (SEQ ID NO: 17).

9. The method of claim 1, wherein the microorganism is a virus.

10. The method of claim 9, wherein the nucleic acid sequence is selected from the group consisting of CCA AGC TAA GAY CGT TTG GTG CCT TGG CTT GG (SEQ ID NO: 12); CGC GAT AAC AGC CTG AAG GAA GCA ACG AAA TCG CG (SEQ ID NO: 13); CGC GAT CTA CCA GCA GCG CCA GAC GGA TCG CG (SEQ ID NO: 20); CGC GAT ATC CTC TCC TTT GCA CGC CGT GGG ACC ATA TCG CG (SEQ ID NO: 21); and combinations thereof.

10. The method of claim 9, wherein the virus is influenza A virus and the nuclic acid sequence comprises CCA AGC TAA GAY CGT TTG GTG CCT TGG CTT GG (SEQ ID NO: 12).

11. The method of claim 9, wherein the virus is SARS coronavirus and the nucleic acid sequence comprises CGC GAT AAC AGC CTG AAG GAA GCA ACG AAA TCG CG (SEQ ID NO: 13).

12. The method of claim 1, further comprising the step of quantitating the microorganism.

13. The method of claim 12, further comprising the step of collecting the microorganism on a filter.

14. The method of claim 1, wherein the microorganism is substantially intact.

15. The method of claim 1, wherein the signal is a fluorescence signal.

16. A method for unamplified, selective identification of methicillin-resistant Staphylococcus aureus comprising:
a) obtaining a sample comprising methicillin-resistant Staphylococcus aureus, wherein the sample comprises unseparated genetic material of the methicillin-resistant Staphylococcus aureus;
b) adding to the sample a molecular beacon comprising a nucleic acid sequence selected from the group consisting of GCG GCT GCT GGC ACG TAG TTA (SEQ ID NO: 1), CCC GCG CGT CGC GAT TTC AAT ATG TAT GCT TTG GTC TTT CTG ATC GCG (SEQ ID NO: 3), and combinations thereof; and
c) heating the sample;
wherein the molecular beacon hybridizes a nucleic acid sequence in the organism of interest to produce a detectable signal.

17. The method of claim 16, wherein the molecular beacon comprises GCG GCT GCT GGC ACG TAG TTA (SEQ ID NO: 1).

18. The method of claim 16, wherein the sample is obtained from the air, a liquid, a surface, soil, an animal, or combinations thereof.

19. The method of claim 16, wherein the detectable signal is a fluorescence signal.

20. The method of claim 19, further comprising the step of quantitating the microorganism.


SEQUENCE LISTING (24 sequences)

SEQ ID NO: 1 - DNA, 21 bp, Staphylococcus aureus
  Resistant Methicillin Staphylococcus aureus
gcggctgctggcacgtagtta

SEQ ID NO: 2 - DNA, 37 bp, Staphylococcus aureus
cccgcgcgtagttactgcgttgtaagacgtccgcggg

SEQ ID NO: 3 - DNA, 39 bp, Staphylococcus aureus
  Resistant Methicillin Staphylococcus aureus
cgcgatttcaatatgtatgctttggtctttctgatcgcg

SEQ ID NO: 4 - DNA, 37 bp, Clostridium difficile
cacgcggattttgaatctcttcctctagtagcgcgtg

SEQ ID NO: 5 - DNA, 40 bp, Acinetobacter baumannii
gcggatagtgtgatctgacgaagacacattaactatcgcg

SEQ ID NO: 6 - DNA, 39 bp, Enterococcus
  Vancomycin-resistant Enterococci (VRE)
cgcgattcgatgagggcggaaaacccaataattatcgcg

SEQ ID NO: 7 - DNA, 36 bp, Streptococcus pneumoniae
cgcgatcaggtctcagcattccaaccgccgatcgcg

SEQ ID NO: 8 - DNA, 26 bp, Salmonella typhimurium
cgctatccggggcgtaacccgtagcg

SEQ ID NO: 9 - DNA, 37 bp, Serratia marcescens
cgcgattgcgctttacgcccagtaattccgaatcgcg

SEQ ID NO: 10 - DNA, 42 bp, Chlamydia trachomatis
ccgtcactgggagaaagaaatggtaggttgttggaatgacgg

SEQ ID NO: 11 - DNA, 29 bp, Mycobacterium
  Multi-drug resistant Mycobacterium
cctggagccgactgttggcgctgtccagg

SEQ ID NO: 12 - DNA, 32 bp, Influenza A virus
ccaagctaagaycgtttggtgccttggcttgg

SEQ ID NO: 13 - DNA, 35 bp, Coronavirus
  SARS Coronavirus
cgcgataacagcctgaaggaagcaacgaaatcgcg

SEQ ID NO: 14 - DNA, 37 bp, Plasmodium falciparum
ctggccctggaggagtattaatgttattatcggccag

SEQ ID NO: 15 - DNA, 37 bp, Escherichia coli
  strain O157H7
cgctatggtgaaggtggaatggttgtcacgaatagcg

SEQ ID NO: 16 - DNA, 33 bp, Enterobacter aerogenes
cgcgatcagacgaagtggtcatgcttgatcgcg

SEQ ID NO: 17 - DNA, 29 bp, Bacillus anthracis
ccgacgagggttgtcagaggatgcgtcgg

SEQ ID NO: 18 - DNA, 37 bp, Yersinia pestis
cgcgattgtctgtttcccatagatgccatgaatcgcg

SEQ ID NO: 19 - DNA, 32 bp, Francisella Tularensis
cgctcgtggagtcggtgtaaaggctccgagcg

SEQ ID NO: 20 - DNA, 32 bp, Ebola virus
cgcgatctaccagcagcgccagacggatcgcg

SEQ ID NO: 21 - DNA, 41 bp, Aphtho virus A
  hoof and mouth disease
cgcgatatcctctcctttgcacgccgtgggaccatatcgcg

SEQ ID NO: 22 - DNA, 35 bp, Xylella fastidiosa
cgcgataaccgcagcagaagccgctcatcatcgcg

SEQ ID NO: 23 - DNA, 36 bp, Burkholderia mallei
cgctgcgttggggattcatttccttagtaagcagcg

SEQ ID NO: 24 - DNA, 35 bp, Bacillus subtilis
cgcgatctaactacgtgccagcagccgcgatcgcg